(19) United States
(12) Patent Application Publication
(10) Pub. No.: US 2018/0150929 A1
(43) Pub. Date: May 31, 2018

(54) METHOD AND SYSTEM FOR REGISTRATION OF 2D/2.5D LAPAROSCOPIC AND ENDOSCOPIC IMAGE DATA TO 3D VOLUMETRIC IMAGE DATA

(71) Applicant: Siemens Aktiengesellschaft, Munich (DE)

(72) Inventors: Thomas PHEIFFER, Langhorne, PA (US); Stefan KLUCKNER, Berlin (DE); Peter MOUNTNEY, London (GB); Ali KAMEN, Skillman, NJ (US)

(21) Appl. No.: 15/570,393

(22) PCT Filed: May 11, 2015
(86) PCT No.: PCT/US2015/030080

§ 371 Date: Oct. 30, 2017

(51) Int. Cl. G06T 3/00; G06T 7/32; G06T 7/00; A61B 34/10

(52) U.S. Cl. CPC G06T 3/0068; G06T 7/32; G06T 7/0014; G06T 7/11; G06T 2207/30056; G06T 2200/04; G06T 2207/10068; A61B 34/10

(57) ABSTRACT

A method and system for registration of 2D/2.5D laparoscopic or endoscopic image data to 3D volumetric image data is disclosed. A plurality of 2D/2.5D intra-operative images of a target organ are received, together with corresponding relative orientation measurements for the intraoperative images. A 3D medical image volume of the target organ is registered to the plurality of 2D/2.5D intra-operative images by calculating pose parameters to match simulated projection images of the 3D medical image volume to the plurality of 2D/2.5D intra-operative images, and the registration is constrained by the relative orientation measurements for the intra-operative images.

### BACKGROUND OF THE INVENTION

The present invention relates to registration of laparoscopic or endoscopic image data to 3D volumetric image data, and more particularly, to registering intra-operative 2D/2.5D laparoscopic or endoscopic image data to pre-operative 3D volumetric image data in order to overlay information from the pre-operative 3D volumetric image data on the intra-operative laparoscopic or endoscopic image data.

During minimally invasive surgical procedures, sequences of laparoscopic or endoscopic images are acquired to guide the surgical procedures. Multiple 2D images can be acquired and stitched together to reconstruct a 3D intra-operative model of an observed organ of interest. This reconstructed intra-operative model may then be fused with pre-operative or intra-operative volumetric image data, such as magnetic resonance (MR), computed tomography (CT), or positron emission tomography (PET), to provide additional guidance to a clinician performing the surgical procedure. However, registration is challenging due to a large parameter space and a lack of constraints on the registration problem. One strategy for performing this registration is to attach the intra-operative camera to an optical or electromagnetic external tracking system in order to establish the absolute pose of the camera with respect to the patient. Such a tracker-based approach does help establish an initial registration between the intra-operative image stream (video) and the volumetric image data, but introduces the burden of additional hardware components to the clinical workflow.

### BRIEF SUMMARY OF THE INVENTION

The present invention provides a method and system for registration of intra-operative images, such as laparoscopic or endoscopic images, with pre-operative volumetric image data. Embodiments of the present invention register a 3D volume to 2D/2.5D intra-operative images by simulating virtual projection images from the 3D volume according to a viewpoint and direction of a virtual camera, and then calculate registration parameters to match the simulated projection images to the real intra-operative images while constraining the registration using relative orientation measurements associated with the intra-operative images from orientation sensors, such as gyroscopes or accelerometers, attached to the intra-operative camera. Embodiments of the present invention further constrain the registration based on a priori information of a surgical plan.

In one embodiment of the present invention, a plurality of 2D/2.5D intra-operative images of a target organ and corresponding relative orientation measurements for the intraoperative images are received. A 3D medical image volume of the target organ is registered to the plurality of 2D/2.5D intra-operative images by calculating pose parameters to match simulated projection images of the 3D medical image volume to the plurality of 2D/2.5D intra-operative images, wherein the registration is constrained by the relative orientation measurements for the intra-operative images.

These and other advantages of the invention will be apparent to those of ordinary skill in the art by reference to the following detailed description and the accompanying drawings.

### BRIEF DESCRIPTION OF THE DRAWINGS

FIG. 1 illustrates a method for registering a 3D pre-operative medical image volume of a target anatomical object to 2D/2.5D intra-operative images of the target anatomical object, according to an embodiment of the present invention;

FIG. 2 illustrates an example of matching simulated projection images from a pre-operative 3D medical image volume to intra-operative images;

FIG. 3 illustrates a method for surgical planning and registration of a 3D pre-operative medical image volume of a target anatomical object to intra-operative images of the target anatomical object, according to an embodiment of the present invention;

FIG. 4 illustrates exemplary constraints determined from a priori knowledge resulting from a surgical plan; and

FIG. 5 is a high-level block diagram of a computer capable of implementing the present invention.

### DETAILED DESCRIPTION

The present invention relates to a method and system for registering intra-operative images, such as laparoscopic or endoscopic images, to 3D volumetric medical images. Embodiments of the present invention are described herein to give a visual understanding of the registration method. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

The fusion of 3D medical image data with an intra-operative image (e.g., frame of endoscopic or laparoscopic video) can be performed by first performing an initial rigid alignment and then a more refined non-rigid alignment. Embodiments of the present invention provide the rigid registration between the 3D volumetric medical image data and the intra-operative image data using sparse relative orientation data from an accelerometer or gyroscope attached to the intra-operative camera, as well as surgical planning information, to constrain an optimization for registration parameters which best align the observed intra-operative image data with simulated projections of a 3D pre-operative medical image volume. Embodiments of the present invention further provide an advantageous surgical planning workflow in which surgical planning information can be used in a biomechanical model to predict motion of tissue in a surgical plan, which is used to provide feedback to the user with respect to a predicted registration quality and guidance on what changes can be made to the surgical plan in order to improve the registration.

Embodiments of the present invention perform co-registration of a 3D pre-operative medical image volume and 2D intra-operative images, such as laparoscopic or endoscopic images, having corresponding 2.5D depth information associated with each image. Is to be understood that the terms “laparoscopic image” and “endoscopic image” are used interchangeably herein and the term “intra-operative image” refers to any medical image data acquired during a surgical procedure or intervention, including laparoscopic images and endoscopic images.

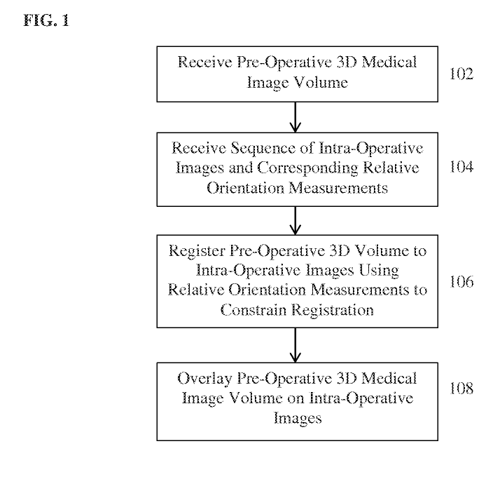

FIG. 1 illustrates a method for registering a 3D pre-operative medical image volume of a target anatomical object to 2D/2.5D intra-operative images of the target anatomical object, according to an embodiment of the present invention. The method of FIG. 1 transforms intra-operative image data representing a patient's anatomy to perform semantic segmentation of each frame of the intra-operative image data and generate a 3D model of a target anatomical object. In an exemplary embodiment, the method of FIG. 1 can be used to register a pre-operative 3D medical image volume in which the liver has been segmented to frames of an intra-operative image sequence of the liver for guidance of a surgical procedure on the liver, such as a liver resection to remove a tumor or lesion from the liver.

Referring to FIG. 1, at step 102, a pre-operative 3D medical image volume is received. The pre-operative 3D medical image volume is acquired prior to the surgical procedure. The 3D medical image volume can be acquired using any imaging modality, such as computed tomography (CT), magnetic resonance (MR), or positron emission tomography (PET). The pre-operative 3D medical image volume can be received directly from an image acquisition device, such as a CT scanner or MR scanner, or can be received by loading a previously stored 3D medical image volume from a memory or storage of a computer system. In a possible implementation, in a pre-operative planning phase, the pre-operative 3D medical image volume can be acquired using the image acquisition device and stored in the memory or storage of the computer system. The pre-operative 3D medical image can then be loaded from the memory or storage system during the surgical procedure.

The pre-operative 3D medical image volume includes a target anatomical object, such as a target organ. In an advantageous implementation, the target organ can be the liver. The pre-operative volumetric imaging data can provide for a more detailed view of the target anatomical object, as compared to intra-operative images, such as laparoscopic and endoscopic images. The target anatomical object and other anatomical objects can be segmented in the pre-operative 3D medical image volume. Surface targets (e.g., liver), critical structures (e.g., portal vein, hepatic system, biliary tract, and other targets (e.g., primary and metastatic tumors) may be segmented from the pre-operative imaging data using any segmentation algorithm. For example, the segmentation algorithm may be a machine learning based segmentation algorithm. In one embodiment, a marginal space learning (MSL) based framework may be employed, e.g., using the method described in U.S. Pat. No. 7,916,919, entitled “System and Method for Segmenting Chambers of a Heart in a Three Dimensional Image,” which is incorporated herein by reference in its entirety. In another embodiment, a semi-automatic segmentation technique, such as, e.g., graph cut or random walker segmentation can be used.

At step 104, a sequence of intra-operative images is received along with corresponding relative orientation measurements. The sequence of intra-operative images can also be referred to as a video, with each intra-operative image being a frame of the video. For example, the intra-operative image sequence can be a laparoscopic image sequence acquired via a laparoscope or an endoscopic image sequence acquired via an endoscope. According to an advantageous embodiment, each frame of the intra-operative image sequence is a 2D/2.5D image. That is each frame of the intra-operative image sequence includes a 2D image channel that provides typical 2D image appearance information for each of a plurality of pixels and a 2.5D depth channel that provides depth information corresponding to each of the plurality of pixels in the 2D image channel. For example, each frame of the intra-operative image sequence can include RGB-D (Red, Green, Blue+Depth) image data, which includes an RGB image, in which each pixel has an RGB value, and a depth image (depth map), in which the value of each pixel corresponds to a depth or distance of the considered pixel from the camera center of the image acquisition device (e.g., laparoscope or endoscope). The intra-operative image acquisition device (e.g., laparoscope or endoscope) used to acquire the intra-operative images can be equipped with a camera or video camera to acquire the RGB image for each time frame, as well as a time of flight or structured light sensor to acquire the depth information for each time frame. The intra-operative image acquisition device can also be equipped with an orientation sensor, such as an accelerometer or a gyroscope, which provides a relative orientation measurement for each of the frames. The frames of the intra-operative image sequence may be received directly from the image acquisition device. For example, in an advantageous embodiment, the frames of the intra-operative image sequence can be received in real-time as they are acquired by the image acquisition device. Alternatively, the frames of the intra-operative image sequence can be received by loading previously acquired intra-operative images stored on a memory or storage of a computer system.

According to an embodiment of the present invention, the sequence of intra-operative images can be acquired by a user (e.g., doctor, clinician, etc.) performing a complete scan of the target organ using the image acquisition device (e.g., laparoscope or endoscope). In this case the user moves the image acquisition device while the image acquisition device continually acquires images (frames), so that the frames of the intra-operative image sequence cover the complete surface of the target organ. This may be performed at a beginning of a surgical procedure to obtain a full picture of the target organ at a current deformation. A 3D stitching procedure may be performed to stitch together the intra-operative images to form an intra-operative 3D model of the target organ, such as the liver.

At step 106, the pre-operative 3D medical image volume is registered to the 2D/2.5D intra-operative images using the relative orientation measurements of the intra-operative images to constrain the registration. According to an embodiment of the present invention, this registration is performed by simulating camera projections from the pre-operative 3D volume using a parameters space defining the position and orientation of a virtual camera (e.g., virtual endoscope/laparoscope). The simulation of the projection images from the pre-operative 3D volume can include photorealistic rendering. The position and orientation parameters determine the appearance and well as the geometry of simulated 2D/2.5D projection images from the 3D medical image volume, which are directly compared to the observed 2D/2.5D intra-operative images via a similarity metric.

An optimization framework is used to select the pose parameters for the virtual camera that maximize the similarity (or minimize the difference) between the simulated projection images and the received intra-operative images. That is, the optimization problem calculates position and orientation parameters that maximize a total similarity (or minimizes a total difference) between each 2D/2.5 intra-operative image and a corresponding simulated 2D/2.5D projection image from the pre-operative 3D volume over all of the intra-operative images. According to an embodiment of the present invention, the similarity metric is calculated for the target organ in intra-operative images and the corresponding simulated projection images. This optimization problem can be performed using any similarity or difference metric and can be solved using any optimization algorithm. For example, the similarity metric can be cross correlation, mutual information, normalized mutual information, etc., and the similarity metric may be combined with a geometry fitting term for fitting the simulated 2.5D depth data to the observed 2.5D depth data based on the geometry of the target organ. As described above the orientation sensors mounted to the intra-operative image acquisition device (e.g., endoscope/laparoscope) provide relative orientations of the intra-operative images with respect to each other. These relative orientations are used to constrain the optimization problem. In particular, the relative orientations of the intra-operative images constrain the set of orientation parameters calculated for the corresponding simulated projection images. Additionally, the scaling is known due to metric 2.5D sensing, resulting in an optimization for pose refinement on the unit sphere. The optimization may be further constrained based on other a priori information from a known surgical plan used in the acquisition of the intra-operative images, such as a position of the operating room table, position of the patient on the operating room table, and a range of possible camera orientations.

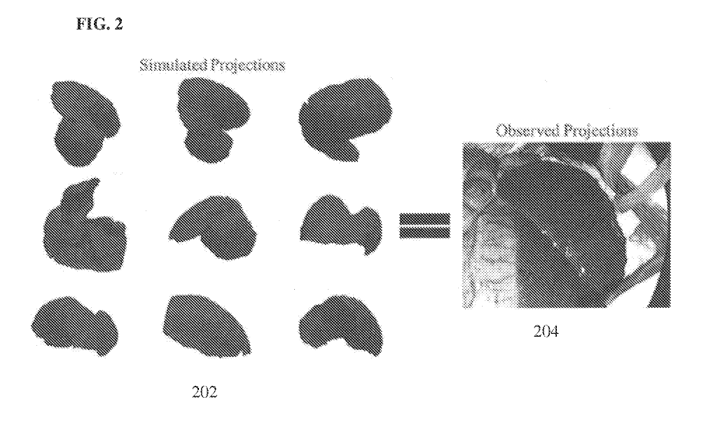

FIG. 2 illustrates an example of matching simulated projection images from a pre-operative 3D medical image volume to intra-operative images. As shown in FIG. 2, image 202 shows a number of simulated 2D projections of the liver generated from a pre-operative 3D medical image volume in which the liver was segmented, and image 204 shows an observed 2D projection of the liver in a laparoscopic image. The registration procedure finds position and orientation parameters to best match a simulated projection of the target organ to each observed projection of the target organ.

Returning to FIG. 1, at step 108, the pre-operative 3D medical image volume is overlaid on intra-operative images during the surgical procedure. The result of the registration is a transformation matrix that can be applied to the pre-operative 3D medical image volume to map a projection of the pre-operative 3D medical image volume onto a given intra-operative image. This enables augmented reality overlays of subsurface information from the pre-operative 3D medical image volume onto the visual information from the intra-operative image acquisition device (e.g., endoscope or laparoscope). In an advantageous embodiment, once the registration is performed new frames of the intra-operative image sequence (video) are received and the projection of the target organ from the pre-operative 3D medical image volume is overlaid on each new frame based on the registration. Each frame including the overlaid information from the pre-operative 3D medical image volume is displayed on a display device to guide the surgical procedure. The overlay can be performed in real-time as the intra-operative images are acquired, and the overlaid images can be displayed on the display device as a video stream. As the registration described herein is a rigid registration, in a possible implementation, a biomechanical model of the target organ may be used to calculate non-rigid deformation of the target organ for each frame. The calculation of the non-rigid deformation using the biomechanical model is described in greater detail in International Patent Application No. PCT/US2015/28120, entitled “System and Method for Guidance of Laparoscopic Surgical Procedures through Anatomical Model Augmentation”, filed Apr. 29, 2015, the disclosure of which is incorporated herein by reference in its entirety.

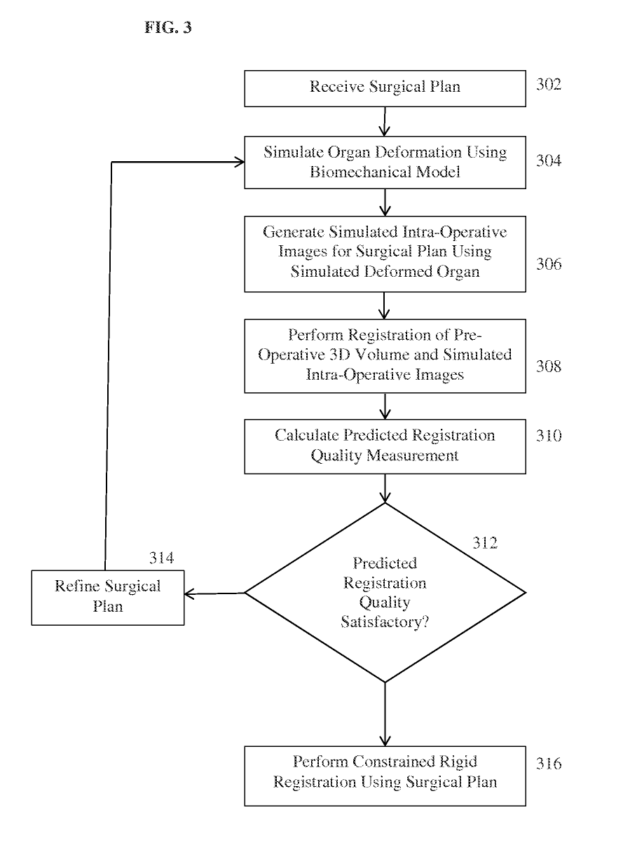

FIG. 3 illustrates a method for surgical planning and registration of a 3D pre-operative medical image volume of a target anatomical object to intra-operative images of the target anatomical object, according to an embodiment of the present invention. The method of FIG. 3 utilizes a surgical planning module that can be implemented on a computer, such as a workstation in an operating room. At step 302, a surgical plan is received. Using the surgical planning module, a user can designate a region of the target organ corresponding to the anticipated intra-operative camera view. For example a 3D surface rendering of the target organ may be shown on a computer display with tools provided for the user to adjust the viewing angle and select structural features of interest via a user input device, such as a mouse or touch screen. The 3D surface rendering of the target organ can be automatically generated from the segmentation of the target organ in the pre-operative 3D medical image volume. Additionally, the anticipated location of the laparoscope entry port at the patient surface may also be indicated. Other relevant intra-operative pose parameters, such as the position of the patient on the operating table may also be gathered and recorded in the surgical plan.

At step 304, deformation of the target organ is simulated using a biomechanical model of the segmented organ. In particular, a 3D mesh of the target organ can be generated from the segmented target organ in the pre-operative 3D medical image volume, and a biomechanical model can be used to deform the 3D mesh in order to simulate expected tissue motion of the target organ given the conditions defined in the surgical plan. The biomechanical model calculates displacements at various points of the 3D mesh based on mechanical properties of the organ tissue and forces applied to the target organ due to the conditions of the surgical plan. For example, one such force may be a force due to gas insufflation of the abdomen in the surgical procedure. In a possible implementation, the biomechanical model models the target organ as a homogeneous linear elastic solid whose motion is governed by the elastodynamics equation. The biomechanical model may be implemented as described in International Patent Application No. PCT/US2015/28120, entitled “System and Method for Guidance of Laparoscopic Surgical Procedures through Anatomical Model Augmentation”, filed Apr. 29, 2015, or International Publication No. WO 2014/127321 A2, entitled “Biomechanically Driven Registration of Pre-Operative Image to Intra-Operative 3D Images for Laparoscopic Surgery”, the disclosures of which are incorporated herein by reference in their entirety.

At step 306, simulated intra-operative images for the surgical plan are generated using the simulated deformed target organ. The simulated intra-operative images are generated by extracting a plurality of virtual projection images of the simulated deformed target organ based on the conditions of the surgical plan, such as the designated portion of the organ to view, a range of possible orientations of the intra-operative camera, and the location of the laparoscope entry point. At step 308, rigid registration of the pre-operative 3D medical image volume to the simulated intra-operative images is performed. In particular, the method of FIG. 1, described above can be performed to register the pre-operative 3D medical image to the simulated intra-operative images in order to predict the results of the registration using intra-operative images acquired with the current surgical plan.

At step 310, a predicted registration quality measurement is calculated. In a possible implementation a surface error for predicted registration. In particular, a total surface error between the simulated projection images of the pre-operative 3D volume and the simulated intra-operative images extracted from the simulated deformed target organ can be calculated. In addition other metrics measuring the extent and quality of organ structure features within the intra-operative camera field of view for the current surgical plan can also be calculated. At step 312, it is determined if the predicted registration quality is sufficient. If it is determined that the predicted registration quality is not satisfactory, the method proceeds to step 314. If it is determined that the predicted registration quality is satisfactory, the method proceeds to step 316. In a possible implementation, it can be automatically determined if the predicted registration quality is sufficient, for example by comparing the predicted registration quality measurement (e.g., surface error) to a threshold value. In another possible implementation, the surgical planning module can present the results to the user and the user can decide whether the predicted registration quality is sufficient. For example, the predicted registration quality measurement or multiple predicted registration quality measurements, as well as the deformed target organ resulting from the biomechanical simulation, can be displayed on a display device. In addition to presenting the results of the biomechanical simulation and corresponding registration to the user to help guide the planning process, the surgical planning module may also provide suggestions regarding parameters of the surgical plan, such as port placement and patient orientation, to improve the registration results.

At step 314, if it is determined that the predicted registration quality is not satisfactory, the surgical plan is refined. For example the surgical plan can be refined by automatically adjusting parameters, such as port placement and patient orientation, to improve the registration results, or the surgical plan can be refined by the user manually changing parameters of the surgical plan via user input to the surgical planning module. It is possible, that the user manually changes the parameters of the surgical plan to incorporate suggested changes provided to the user by the surgical planning module. The method then returns to step 304 and repeats steps 304-312 to simulate the deformation of the organ and predict the registration quality for the refined surgical plan.

At step 316, when it is determined that the predicted registration quality for the surgical plan is sufficient, constrained rigid registration is perform using the surgical plan. The registration method of FIG. 1, described above, is further constrained based on a priori knowledge resulting from the surgical plan. In particular, once the surgical plan is finalized, intra-operative images are acquired using the surgical plan, the method of FIG. 1 is used to register the pre-operative 3D medical image volume with the acquired intra-operative images, and the parameters of the surgical plan, such as the patient pose on the operating table and the port placement for laparoscopic images, provide further constraints for the registration.

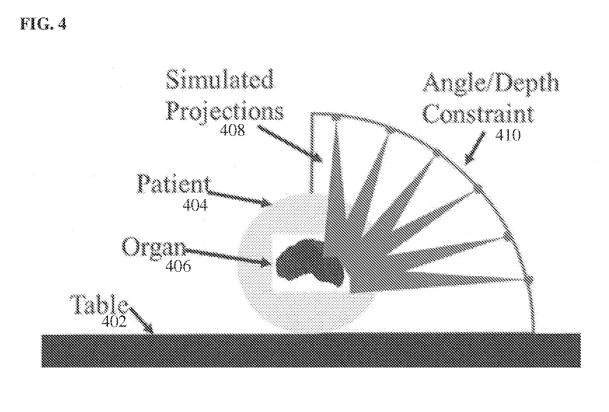

FIG. 4 illustrates exemplary constraints determined from a priori knowledge resulting from a surgical plan. As shown in FIG. 4, a location of the operating room table 402 and a pose of the patient 404 relative to the table 402 are known from the surgical plan. The simulated deformation of the target organ 406 and the simulated projection images 408 (simulated intra-operative images) can provide constraints angle and depth constraints 410 related to a range of angles and depths of the simulated projection images 408 with respect the organ 406 and the patient 404.

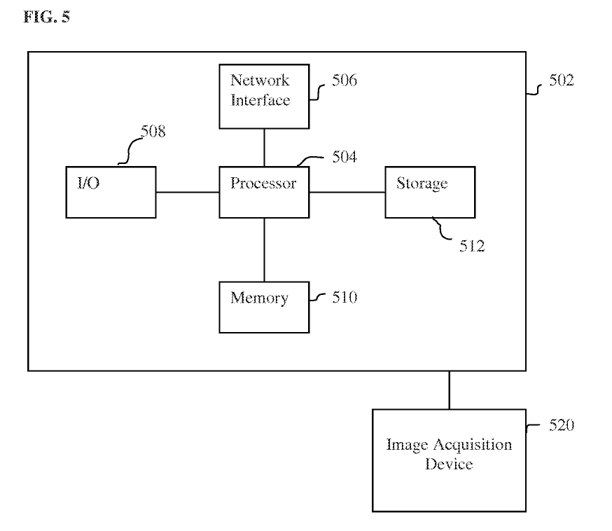

The above-described methods for registering 3D volumetric image data to intra-operative images and for surgical planning to improve such a registration may be implemented on a computer using well-known computer processors, memory units, storage devices, computer software, and other components. A high-level block diagram of such a computer is illustrated in FIG. 5. Computer 502 contains a processor 504, which controls the overall operation of the computer 502 by executing computer program instructions which define such operation. The computer program instructions may be stored in a storage device 512 (e.g., magnetic disk) and loaded into memory 510 when execution of the computer program instructions is desired. Thus, the steps of the methods of FIGS. 1 and 3 may be defined by the computer program instructions stored in the memory 510 and/or storage 512 and controlled by the processor 504 executing the computer program instructions. An image acquisition device 520, such as a laparoscope, endoscope, CT scanner, MR scanner, PET scanner, etc., can be connected to the computer 502 to input image data to the computer 502. It is possible that the image acquisition device 520 and the computer 502 communicate wirelessly through a network. The computer 502 also includes one or more network interfaces 506 for communicating with other devices via a network. The computer 502 also includes other input/output devices 508 that enable user interaction with the computer 502 (e.g., display, keyboard, mouse, speakers, buttons, etc.). Such input/output devices 508 may be used in conjunction with a set of computer programs as an annotation tool to annotate volumes received from the image acquisition device 520. One skilled in the art will recognize that an implementation of an actual computer could contain other components as well, and that FIG. 5 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method for registering a 3D medical image volume of a target organ to 2D/2.5D intra-operative images of the target organ comprising:
receiving a plurality of 2D/2.5D intra-operative images of the target organ and corresponding relative orientation measurements for the intraoperative images; and
registering a 3D medical image volume of the target organ to the plurality of 2D/2.5D intra-operative images by calculating pose parameters to match simulated projection images of the 3D medical image volume to the plurality of 2D/2.5D intra-operative images, wherein the registration is constrained by the relative orientation measurements for the intra-operative images.

2. The method of claim 1, wherein registering a 3D medical image volume of the target organ to the plurality of 2D/2.5D intra-operative images by calculating pose parameters to match simulated projection images of the 3D medical image volume to the plurality of 2D/2.5D intra-operative images, wherein the registration is constrained by the relative orientation measurements for the intra-operative images comprises:
optimizing pose parameters of the simulated projection images of the 3D medical image volume to maximize a similarity metric between each of the plurality of 2D/2.5D intra-operative images and a corresponding one of the simulated projection images of the 3D medical image volume, wherein the pose parameters of the simulated projection images of the 3D medical image volume are constrained by the relative orientation measurements for the intra-operative images.

3. The method of claim 2, wherein the relative orientation measurements are received from an orientation sensor mounted to an intra-operative image acquisition device used to acquire the plurality of intra-operative images, and the relative orientation measurements represent a relative orientation of the intra-operative image acquisition device corresponding to each of the plurality of intra-operative images, wherein the pose parameters of the simulated projection images of the 3D medical image volume comprise virtual camera position and orientation parameters for each of the simulated projection images, and wherein the virtual camera orientation parameters for the simulated projection images are constrained such that relative orientations of the virtual camera for the simulated projection images matches the relative orientations of the plurality of intra-operative images.

4. The method of claim 2, wherein each of the plurality of 2D/2.5D intra-operative images includes 2D image data and corresponding 2.5D depth data, each of the simulated projection images of the 3D medical image volume is a 2D/2.5D projection image including 2D image data and corresponding 2.5D depth data, and optimizing pose parameters of the simulated projection images of the 3D medical image volume to maximize a similarity metric between each of the plurality of 2D/2.5D intra-operative images and a corresponding one of the simulated projection images of the 3D medical image volume, wherein the pose parameters of the simulated projection images of the 3D medical image volume are constrained by the relative orientation measurements for the intra-operative images comprises:
optimizing the pose parameters of the simulated projection images of the 3D medical image volume to maximize a cost function including an appearance based similarity metric between the 2D image data in each of the plurality of 2D/2.5 intra-operative images and the corresponding one of the simulated projection images and a geometry fitting metric between the 2.5D depth data in each of the plurality of 2D/2.5D intra-operative images and the corresponding one of the simulated projection images.

5. The method of claim 1, wherein the registration is further constrained based on a priori information from a known surgical plan used to acquire the plurality of 2D/2.5D intra-operative images.

6. The method of claim 5, wherein the a priori information comprises a pose of a patient relative to an operating room table.

7. The method of claim 1, wherein receiving a plurality of 2D/2.5D intra-operative images of the target organ and corresponding relative orientation measurements for the intraoperative images comprises:
receiving the plurality of 2D/2.5D intra-operative images from an intraoperative image acquisition device, wherein the intra-operative image acquisition device is one of a laparoscope or an endoscope; and
receiving the corresponding relative orientation measurements for the intra-operative images from an orientation sensor mounted to the intra-operative image acquisition device, wherein the orientation sensor is one of a gyroscope or an accelerometer.

8. The method of claim 1, further comprising:
prior to receiving the plurality of 2D/2.5D intra-operative images:
simulating deformation of the target organ based on a surgical plan using a biomechanical model of the target organ;
generating simulated intra-operative images for the surgical plan using the simulated deformation of the target organ;
registering the 3D medical image volume of the target organ to the simulated intra-operative images; and
calculating a predicted registration quality measurement for surgical plan based on the registration of the 3D medical image volume of the target organ to the simulated intra-operative images.

9. The method of claim 8, further comprising:
prior to receiving the plurality of 2D/2.5D intra-operative images, refining parameters of the surgical plan in response to a determination, based on the predicted registration quality measurement, that a predicted registration quality for the surgical plan is insufficient.

10. The method of claim 8, wherein receiving a plurality of 2D/2.5D intra-operative images of the target organ and corresponding relative orientation measurements for the intraoperative images comprises:
receiving the plurality of 2D/2.5D intra-operative images of the target organ acquired using the surgical plan, wherein the registration is further constrained based on one or more parameters of the surgical plan.

11. The method of claim 10, wherein the one or more parameters of the surgical plan comprise at least one of a pose of a patient relative to an operating table, a location of a laparoscopic entry port, or a range of angles of an intra-operative image acquisition device used to acquire the plurality of 2D/2.5D intra-operative images.

12. An apparatus for registering a 3D medical image volume of a target organ to 2D/2.5D intra-operative images of the target organ comprising:
means for receiving a plurality of 2D/2.5D intra-operative images of the target organ and corresponding relative orientation measurements for the intraoperative images; and
means for registering a 3D medical image volume of the target organ to the plurality of 2D/2.5D intra-operative images by calculating pose parameters to match simulated projection images of the 3D medical image volume to the plurality of 2D/2.5D intra-operative images, wherein the registration is constrained by the relative orientation measurements for the intra-operative images.

13. The apparatus of claim 12, wherein the means for registering a 3D medical image volume of the target organ to the plurality of 2D/2.5D intra-operative images by calculating pose parameters to match simulated projection images of the 3D medical image volume to the plurality of 2D/2.5D intra-operative images comprises:
means for optimizing pose parameters of the simulated projection images of the 3D medical image volume to maximize a similarity metric between each of the plurality of 2D/2.5D intra-operative images and a corresponding one of the simulated projection images of the 3D medical image volume, wherein the pose parameters of the simulated projection images of the 3D medical image volume are constrained by the relative orientation measurements for the intra-operative images.

14. The apparatus of claim 13, wherein the relative orientation measurements are received from an orientation sensor mounted to an intra-operative image acquisition device used to acquire the plurality of intra-operative images, and the relative orientation measurements represent a relative orientation of the intra-operative image acquisition device corresponding to each of the plurality of intra-operative images, wherein the pose parameters of the simulated projection images of the 3D medical image volume comprise virtual camera position and orientation parameters for each of the simulated projection images, and wherein the virtual camera orientation parameters for the simulated projection images are constrained such that relative orientations of the virtual camera for the simulated projection images matches the relative orientations of the plurality of intra-operative images.

15. (canceled)

16. (canceled)

17. The apparatus of claim 12, further comprising:
means for simulating deformation of the target organ based on a surgical plan using a biomechanical model of the target organ;
means for generating simulated intra-operative images for the surgical plan using the simulated deformation of the target organ;
means for registering the 3D medical image volume of the target organ to the simulated intra-operative images; and
means for calculating a predicted registration quality measurement for surgical plan based on the registration of the 3D medical image volume of the target organ to the simulated intra-operative images.

18. (canceled)

19. (canceled)

20. A non-transitory computer readable medium storing computer program instructions for registering a 3D medical image volume of a target organ to 2D/2.5D intra-operative images of the target organ, the computer program instructions when executed on a processor cause the processor to perform operations comprising:
receiving a plurality of 2D/2.5D intra-operative images of the target organ and corresponding relative orientation measurements for the intraoperative images; and
registering a 3D medical image volume of the target organ to the plurality of 2D/2.5D intra-operative images by calculating pose parameters to match simulated projection images of the 3D medical image volume to the plurality of 2D/2.5D intra-operative images, wherein the registration is constrained by the relative orientation measurements for the intra-operative images.

21. The non-transitory computer readable medium of claim 20, wherein registering a 3D medical image volume of the target organ to the plurality of 2D/2.5D intra-operative images by calculating pose parameters to match simulated projection images of the 3D medical image volume to the plurality of 2D/2.5D intra-operative images, wherein the registration is constrained by the relative orientation measurements for the intra-operative images comprises:
optimizing pose parameters of the simulated projection images of the 3D medical image volume to maximize a similarity metric between each of the plurality of 2D/2.5D intra-operative images and a corresponding one of the simulated projection images of the 3D medical image volume, wherein the pose parameters of the simulated projection images of the 3D medical image volume are constrained by the relative orientation measurements for the intra-operative images.

22. The non-transitory computer readable medium of claim 21, wherein the relative orientation measurements are received from an orientation sensor mounted to an intra-operative image acquisition device used to acquire the plurality of intra-operative images, and the relative orientation measurements represent a relative orientation of the intra-operative image acquisition device corresponding to each of the plurality of intra-operative images, wherein the pose parameters of the simulated projection images of the 3D medical image volume comprise virtual camera position and orientation parameters for each of the simulated projection images, and wherein the virtual camera orientation parameters for the simulated projection images are constrained such that relative orientations of the virtual camera for the simulated projection images matches the relative orientations of the plurality of intra-operative images.

23. (canceled)

24. (canceled)

25. The non-transitory computer readable medium of claim 20, wherein receiving a plurality of 2D/2.5D intra-operative images of the target organ and corresponding relative orientation measurements for the intraoperative images comprises:
receiving the plurality of 2D/2.5D intra-operative images from an intraoperative image acquisition device, wherein the intra-operative image acquisition device is one of a laparoscope or an endoscope; and
receiving the corresponding relative orientation measurements for the intra-operative images from an orientation sensor mounted to the intra-operative image acquisition device, wherein the orientation sensor is one of a gyroscope or an accelerometer.

26. The non-transitory computer readable medium of claim 20, wherein the operations further comprise:
prior to receiving the plurality of 2D/2.5D intra-operative images:
simulating deformation of the target organ based on a surgical plan using a biomechanical model of the target organ;
generating simulated intra-operative images for the surgical plan using the simulated deformation of the target organ;
registering the 3D medical image volume of the target organ to the simulated intra-operative images; and
calculating a predicted registration quality measurement for surgical plan based on the registration of the 3D medical image volume of the target organ to the simulated intra-operative images.

27. (canceled)

28. (canceled)

29. (canceled)
